(19) United States
(12) Patent Application Publication
(10) Pub. No.: US 2018/0014903 A1
(43) Pub. Date: Jan. 18, 2018

(54) HEAD-MOUNTABLE COMPUTING DEVICE, METHOD AND COMPUTER PROGRAM PRODUCT

(71) Applicant: KONINKLIJKE PHILIPS N.V., EINDHOVEN (NL)

(72) Inventors: Caifeng SHAN, VELDHOVEN (NL); Radu Serban JASINSCHI, NUENEN (NL); Ronaldus Maria AARTS, GELDROP (NL)

(21) Appl. No.: 15/536,683

(22) PCT Filed: Dec. 9, 2015
(86) PCT No.: PCT/EP2015/079042

§ 371 Date: Jun. 16, 2017

(30) Foreign Application Priority Data
Dec. 18, 2014 (EP) 14198927.7

(51) Int. Cl. G06F 1/16; G06F 3/147; G06F 19/00; G02B 27/01; G06F 3/01

(52) U.S. Cl. CPC A61B 90/37; G06F 1/163; G06F 19/3406; G02B 27/0172; G06F 3/147; A61B 2560/0487; A61B 2090/502; A61B 2090/376; A61B 2090/372; A61B 2562/0204; A61B 2090/0805; G02B 2027/0138; G06F 3/011; A61B 2562/0219

(57) ABSTRACT

Disclosed is a head-mountable computing device comprising at least one display module; an interface adapted to receive signals and/or data acquired by one or more sensors and to receive data generated by one or more medical devices operated by a wearer of the head-mountable computing device; a processor adapted to identify the wearer's activity based on the received signals and/or data acquired by the one or more sensors and to control said at least one display module to display assistance information on said display module corresponding to the identified wearer's activity.

### FIELD OF THE INVENTION

The present invention relates to a head-mountable computing device comprising at least one display module and a processor for controlling the at least one display module to display information on the display module in response to an identified wearer's activity.

The present invention further relates to a method for displaying information on a head-mountable computing device in response to an identified wearer's activity.

The present invention further relates to a computer program product for implementing such a method when executed on a processor of such head-mountable computing device.

### BACKGROUND OF THE INVENTION

Head-mountable computing device, a wearable near-to-eye display device worn on the head of a wearer, has been used in research, military and gaming industry. Such head-mountable computing device can generate video and/or audio user assistance information, such as the identification number of an object or the parameters of an object, and provide such user assistance information to the wearer. For instance, in hospital, the vital signs signals derived from the patient monitors can be displayed on a head-mountable computing device worn on the head of a surgeon in the operation room and can support the surgeon to perform an operation.

However, the displayed user assistance information provided by the head-mountable computing device may be excessive or irrelevant to the wearer and therefore may disturb the wearer, especially when the attention of the wearer is fully focused on performing a specific task, such as performing an operation, and it is very risky to be distracted.

US 2014/0152530 discloses a near-to-eye display system comprising a set of cameras attached to the frame of the eyeglass for capturing video images of a scene in a field of view; a microphone receiving inputs from the wearer. The user assistance information displayed on the near-to-eye display system is derived from interactive video analysis and user input from voice via microphone and signals from hand held devices.

### SUMMARY OF THE INVENTION

It is an object of the present invention to provide a head-mountable computing device for display information on the display module in response to an identified wearer's activity.

It is further an object of the present invention to provide a method for displaying information using such a head-mountable module in response to an identified wearer's activity.

It is further an object of the present invention to provide a computer program product for implementing such a method when executed on a processor of such head-mountable computing device.

According to an aspect, there is provided a head-mountable computing device comprising at least one display module; an interface adapted to receive signals and/or data acquired by one or more sensors, and to receive data generated by one or more medical devices operated by a wearer of the head-mountable computing device; a processor adapted to identify the wearer's activity based on the received signals and/or data acquired by the one or more sensors; and to control said at least one display module to display wearer assistance information on said display module corresponding to the identified wearer's activity.

The present invention is based on the idea that the wearer assistance information, e.g. the visual content information which is relevant to current wearer's activity, will be displayed on the display module of the head-mountable computing device in order to avoid the wearer's distraction by the excessive or irrelevant information displayed on the display module when performing a specific task or activity, e.g. performing a surgery operation in an operation room.

The wearer's activity may be identified by one or more sensors for sensing the current activities of the wearer. The sensors may include an optical sensor for capturing one or more images showing, e.g., the current views of the wearer and/or the current devices the wearer is operating. The optical sensor may be, e.g. a camera integrated into the head-mountable computing device carried by the wearer or may be mounted in a fixed position in an operation room. The sensors may include a microphone for capturing the audio or acoustic data indicating, e.g., the instructions or conversations of the wearer. The sensors may also include an accelerometer or gyroscope showing the movement of the wearer's head or body. The microphone and the accelerometer may be integrated into the head-mountable computing device carried by the wearer.

The processor may identify the wearer's activity based on the received signals and/or data acquired by the one or more sensors. The identification of the wearer's activity may be achieved by, e.g. the use of image processing unit and/or audio processing unit of the processor. For instance, when the wearer is performing a surgery operation, the received signals and/or data acquired by the one or more forward-facing optical sensors may show the field of view of the wearer, e.g. using a knife to cut an opening, and the audio or acoustic data may indicate the instruction of the current wearer, or the conversation between the wearer and other people, e.g. his/her assistant. The image processing unit of the processor may analyse the images and identify the current activities of the wearer based on the recognition of the image information, such as recognizing a knife the wearer is using to cut an opening. In addition, the audio processing unit of the processor may analyse the audio or acoustic data and identify the current activities of the wearer based on intercepting or detecting the keywords in the audio or acoustic data, such as “scissors”, “cut”, “heart”, “opening”, etc.

The wearer assistance information displayed on said display module may include the information for supporting the wearer to perform a specific task. For instance, the assistance information for supporting the wearer to performing a surgery operation may include vital signs of patients, such as SpO₂, ECG, EEG, Respiration Rate, Heart Rate, the temperature of the patient, or medical images of the patient acquired by various acquisition modalities, such as standard X-ray Imaging, Computed Tomography (CT), Magnetic Resonance Imaging (MRI), Ultrasound (US), Positron Emission Tomography (PET), Single Photon Emission Computed Tomography (SPECT), and Nuclear Medicine (NM). Such assistance information may be acquired by medical monitors or devices connected or communicated to the head-mountable device via a transmitter such that the assistance information may be transmitted to head-mountable device in real time. Alternatively, the assistance information may be stored in a remote database. This has the advantage that the wearer may perform a certain activity, such as performing a surgery operation, and simultaneously may hands-freely access to an object's data, such as a patient's vital signs, so as to monitor the status of the object. Alternatively, the wearer assistance information displayed on the display module may be controlled to be automatically hidden, blurred or partly removed from the display module by the processor when a specific activity of the wearer is identified. For instance, when the wearer of the head-mountable device is performing a task that needs full attention from the wearer or engaging an activity that she/he should not be distracted (for example, inserting a catheter into a patient's body for interventional therapy), all the assistance information displayed on the display module will be automatically hidden or blurred by the processor in order to avoid any distraction to the wearer. Alternatively, the hidden or removed assistance information may be provided by the head-mountable computing device in a subtle audio or tactile stimulus manner.

In an embodiment, the processor is adapted to control said at least one display module to display a warning message on said display module based on a predefined threshold of the one or more received data or signals, e.g. the vital signs of a patient, or based on the identification of an abnormal or abrupt state changes of a patient, e.g. bleeding. This has the advantage that it may prevent the user from overlooking any critical changes with the patient. Meanwhile, it allows the wearer to react to the changes without having to take his/her eyes off an object, such as a patient, or an activity that he/she is performing. The predefined threshold of the one or more received data may indicate an abnormality of, or a significant change of the received data or signals, such as the vital signs of the patient. The threshold may be predetermined by the wearer, or may be adaptively adjusted, e.g., based on the conditions of the patient, or learned from the recorded patient data. The warning message may include an alert sound signal, a text or visual content information. The warning message may be popped up to the display module, or may be displayed in the display module with a different colours/brightness or different font/format, in a highlighted or amplified manner other than other displayed information so as to attract the attention of the wearer. The popped up warning message may disappear when a predetermined time period elapses. The predetermined time period may be 5 seconds or 10 seconds depending on the wearer's activity, which is long enough to attract the attention of the wearer. Note that the warning message may be displayed on the display module even if all the assistance information are hidden or removed from the display module based on an identified wearer's activity, such as inserting a catheter into a human body.

In an embodiment, the wearer's activity is identified based on the received signals and/or the data acquired by the one or more sensors in combination with the data received from the one or more medical devices operated by the wearer of the head-mountable computing device. This has the advantage that it enables a precise identification of the wearer's activity. The sensors system for detecting the wearer's activities may have its limitation. For some applications, the wearer's activities may not be detectable, or may be difficult to be detected solely by the sensors. For instance, the optical sensor may detect an object, such as a medical device, that the wearer is observing or is operating, but it may not be detectable how the wearer is operating or manipulating the object, and/or on which part of the object the wearer is operating solely by the optical sensor or audio sensor. In a specific example, when the wearer is operating a medical system/device for performing a diagnosis or treatment procedure, it may be very difficult for the camera/microphones to detect the wearer's activity in detail. As illustrated in FIG. 1, when the wearer performs an RF ablation with the catheter inserted into the patient's body, the wearer's activities such as the manipulation of a catheter, start/stop of an interventional X-ray machine by controlling a pedal of the X-ray machine, start/stop of an ablation, etc., may not be detectable by the camera/microphones due to the fact that such activities may be too short to be captured by the camera/microphones and therefore may be difficult to be identified, or may be hindered or blocked by an obstacle, such as the interventional X-ray machine. In an embodiment, the data received from the one or more medical devices may comprise the identification information of the medical devices. By receiving data from the catheter and/or the interventional X-ray machine operated by the wearer, the processor may identify which medical devices the wearer is currently operating. Moreover, the received data may also indicate on which part of medical devices the wearer is operating, such as the pedal of the X-ray machine. Therefore, the wearer's activities, such as operating on an interventional X-ray machine by controlling the pedal of the X-ray machine, may be precisely identified.

In an embodiment, the wearer assistance information displayed on said display module may include the duration of an operation or activity that the wearer is operated on said one or more medical devices. This has the advantage that it enables the wearer to hand-freely monitor the time spent on his/her current activity.

Alternatively, a warning message may be displayed on the display module when the duration of an operation or activity performed by the wearer is longer than a predefined value. This has the advantage that it enables the wearer to abort the overtime activities or operations he/she is currently performing so as to avoid risks or damage raised by the overtime activities. For instance, an ablation operation may last for a reasonable duration when energy is applied at an appropriate intensity. If the duration of the ablation operation is too short, the lesion may not be transmural, i.e. the disease may not be treated properly. If the duration of the ablation operation is too long, the ablation may cause damage to the neighbouring organs such as lung and esophagus, which may be dangerous and may cause life threatening to the patient. Therefore, it may be critical that the duration of ablation operation may not last for too long. In an embodiment, the information on the duration of an operation or activity may be highlighted, such as using different colours/brightness or different font/format, in order to alert the wearer. Alternatively, the wearer assistance information, such as the ECG or other vital signs of the patient, may be hidden, blurred or partly removed from the display module in order to emphasize the warning message.

According to another aspect, there is provided a method of displaying information on the head-mountable computing device according to one or more of the above embodiments, the method comprising receiving signals and/or data acquired by one or more sensors; receiving one or more signals generated by one or more medical devices operated by a wearer of the head-mountable computing device; identifying the wearer's activity based on the received signals and/or data acquired by the one or more sensors; displaying information on said display module corresponding to the identified wearer's activity. This allows the wearer to avoid the wearer's distraction by the excessive or irrelevant information displayed on the display module when performing a specific task or activity, e.g. performing a surgery operation in an operation room.

In an embodiment, displaying information on the head-mountable computing device may comprise hiding information displayed on said display module corresponding to the identified wearer's activity.

In an embodiment, the identification of the wearer's activity may be based on the received signals and/or the data acquired by the one or more sensors in combination with the data received from the one or more medical devices operated by the wearer of the head-mountable computing device.

In an embodiment, displaying information on the head-mountable computing device may comprise displaying a warning message on said display module based on a predefined threshold of the one or more received signals.

In an embodiment, the wearer assistance information displayed on said display module may include the duration of an operation that the wearer is operated on said one or more medical devices, and a warning message is displayed on the display module when the duration of the operation is longer than a predefined value.

In accordance with yet another aspect, there is provided a computer program product comprising a computer program code for, when executed on the processor of the head-mountable computing device according to one or more of the above embodiments, implementing the steps of the method according to one or more of the above embodiments. Such a computer program product may be made available to the head-mountable computing device in any suitable form, e.g. as a software application (app) available in an app store, and may be used to configure the head-mountable computing device such that the head-mountable computing device may implement the aforementioned method.

### BRIEF DESCRIPTION OF THE DRAWINGS

Embodiments of the invention are described in more detail and by way of non-limiting examples with reference to the accompanying drawings, wherein

FIG. 1 shows a prior art of an activity that a surgeon is performing;

FIG. 2 schematically depicts a head-mountable computing device according to an embodiment;

FIG. 3 shows a flow chart of a first embodiment of a method according to the present invention;

FIG. 4 schematically depicts an aspect of the head mountable computing device of FIG. 2 in more detail;

FIG. 5 shows a flow chart of a second embodiment of a method according to the present invention.

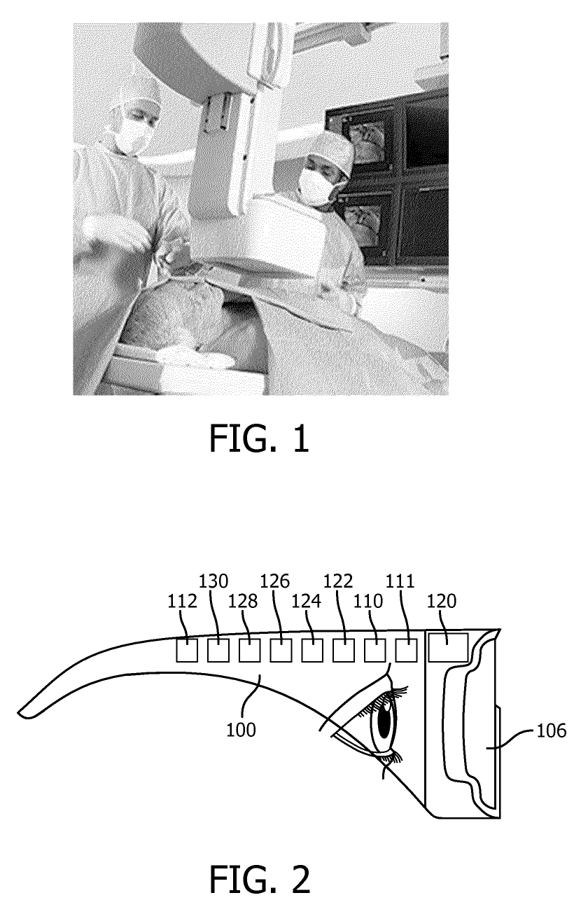

### DETAILED DESCRIPTION OF THE EMBODIMENTS

It should be understood that the Figures are merely schematic and are not drawn to scale. It should also be understood that the same reference numerals are used throughout the Figures to indicate the same or similar parts.

In the context of the present application, a head-mountable computing device is a device that can be worn on the head of its user and provides the user with computing functionality. The head-mountable computing device may be configured to perform specific computing tasks as specified in a software application (app) that may be retrieved from the Internet or another computer-readable medium. Non-limiting examples of such head-mountable computing devices include smart headgear, e.g. eyeglasses, goggles, a helmet, a hat, a visor, a headband, a Google Glass, or any other device that can be supported on or from the wearer's head, and so on.

In the context of the present application, the head-mountable computing device may comprise an optical sensor, such as a camera, for capturing an image in a field of view of a wearer of the wearable computing device. The camera may be arranged such that when the head-mountable computing device is worn as intended, the camera aligns with the eyes of its wearer, i.e. produces a forward-facing sensor signal corresponding to the field of view of its wearer. Such a camera may be integral to the head-mountable computing device. Alternatively, the optical sensor may be mounted in a fixed position, such as in a fixed position of an operation room, and is communicatively coupled via a wired or wireless connection to the head-mountable computing device.

FIG. 2 schematically shows a head-mountable computing device 100 according to an embodiment. The head-mountable computing device 100 may comprise at least one display module 106, which may be a see-through or transparent display module 106, under the control of a processor 110 of the head-mountable computing device 100. The at least one display module 106 is typically arranged such that a wearer of the head-mountable computing device 100 can observe an image or data displayed on the at least one display module 106. Preferably, the at least one display module 106 is a see-through or transparent display module such that the wearer can observe at least a part of a field of view through the display module 106.

The head-mountable computing device 100 may further comprise a camera 120 for capturing an image or video data or signals in a field of view of a wearer of the head-mountable computing device 100. Such captured image or video data or signals may be used for analyzing the activity of the wearer. Alternatively, other electronic devices including a camera may be used for capturing the image or video data or signals from an area including the wearer of the wearable computing device. Examples of such electronic devices may be hand-held devices such as a smartphone, a tablet, etc., other head-mountable computing device, a surveillance device such as an alarm camera mounted in a room, or a vital sign monitoring device such as a device including a vital signs camera used for remote photo-plethysmography.

The head-mountable computing device 100 may further comprise an interface 111 for receiving one or more images or video data captured by the camera 120 or other electronic devices including a camera. The interface 111 may be also used for receiving data from one or more medical devices operated by the wearer of the head-mountable computing device 100.

The head-mountable computing device 100 may further comprise a button 122 as user interface or control element for accepting the wearer's commands, a microphone 124 for capturing the wearer's spoken instruction or the conversations between the wearer and other people, such as his/her assistant, a speaker 126 for allowing the wearer to hear the audio information generated by the head-mountable computing device 100, an accelerometer 130 for recognizing the gesture or the movement of the wearer, such as a turn or nod of the wearer's head, and a transmitter 128.

The processor 110 may execute instructions, i.e. computer program code, stored in a non-transitory computer readable medium, such as data storage 112. The processor 110 may comprise an imaging processing unit 132 for analysing the one or more images or video data or signals received from the interface 111 or directly from the camera 120 to identify the activity of the wearer of the head-mountable computing device 100. The processor may further comprise an audio processing unit 134 for analysing the audio or acoustic data derived from the microphone 124 to identify the current activities of the wearer. The data storage 112 may function as (part of) a database for storing the assistance information, such as the physiological parameters of the patient, the medical images of the patient with diverse modalities, e.g. CT, MRI or X-ray medical images, a Look-Up-Table (LUT) defining the types assistance information needed for each wearer's activity. In an embodiment, the database or portion thereof in the data storage 112 is a local copy that may be synchronized with a remote master database via the transmitter 128. Alternatively, the head-mountable computing device 100 may be adapted to wirelessly communicate with a remote system 200, e.g. a remote server hosting a remote database such as a patient database, via the transmitter 128. Any suitable wireless communication protocol may be used for any of the wireless communication between the head-mountable computing device 100 and the remote system 200, e.g., an infrared link, Zigbee, Bluetooth, a wireless local area network protocol such as in accordance with the IEEE 802.11 standards, a 2G, 3G or 4G telecommunication protocol, and so on.

The button 122 may allow the wearer to select the user instruction from a list of options. Such a list of options for instance may be displayed on the display module 106 of the head-mountable computing device 100.

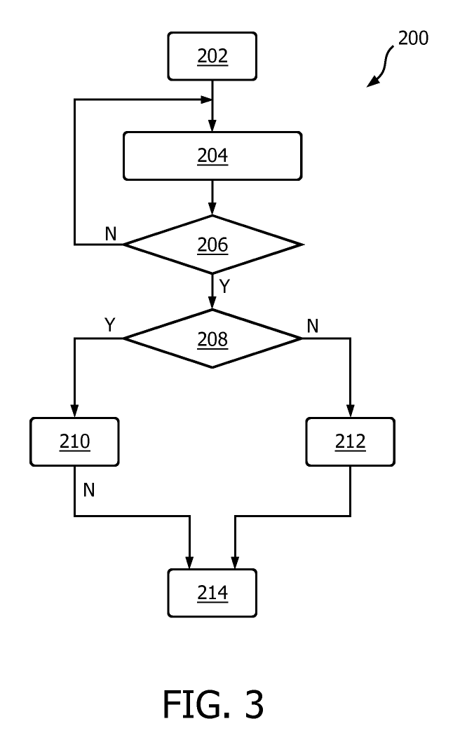

FIG. 3 shows a flow chart of a method 200 for displaying assistance information on the display module 106 of the head-mountable computing device 100 in response to the wearer's activity.

The method 200 starts in step 202 with receiving a starting command regularly triggered by the processor 110. The interval between the two starting commands triggered by the processor 110 may be predetermined by the wearer of the head-mountable computing device 100. Alternatively, the starting command may be triggered by the instruction of the wearer. Such instruction may be provided in any suitable manner, such as by a head movement or gesture via the accelerometer 130, spoken instruction via the microphone 124, or pressing the button 122.

The method 200 then proceeds to step 204 in which one or more images or video data is captured by the camera 120, the audio or acoustic data is captured by the microphone 124, or the data derived from the one or more medical devices is received.

Upon capturing the one or more images or video data, the processor 110 then proceeds to step 206 to identify the activity of the wearer of the head-mountable computing device based on the analysed result of the captured images or video data or signal and/or the captured audio or acoustic data. The activity identification may be achieved, for instance, by detecting the objects in the captured images or video data and comparing the identified objects against a Look-Up-Table (LUT) defining the types of assistance information for each wearer's activity, e.g. a list of objects corresponding to a specific activity in which the needed types of assistance information is predefined. If the detected objects in the captured images or video date are identical to the same list of objects in the LUT corresponding to a specific activity, then such activity is identified. In addition to object detection, the user's activity can also be identified by analysing the movement patterns of the detected object in the video, for example, the wearer's hand movements. Common video analytics methods, such as pattern recognition algorithms, may be used for detecting the objects or the movement patterns of the detected object in the captured images or video data. For instance, the movement pattern of the wearer's hand can be derived, by tracking the hand in the video to derive the trajectory of the motion. There are various visual tracking techniques in the literature can be used to track the detected object(s). For example, a publication titled “Object Tracking: A survey” by Yilmaz, et al ACM Computing Surveys, Vol. 38, Issue 4, 2006, describes the state-of-the-art object tracking methods.

Alternatively, the wearer's activity identification may be achieved, for instance, by detecting the audio or acoustic data and identifying the current activities of the wearer based on intercepting or detecting the keywords in the audio/acoustic data, such as “scissors”, “cut”, “heart”, “opening”, etc. The detected keywords may be compared against a list of keywords in the LUT. Each activity in the LUT may link to one or more keywords. If the detected keywords are identical to the same keywords in the LUT corresponding to an activity, then such activity is identified.

The activity identification may be also achieved by receiving the data acquired from the one or more medical devices operated by the wearer of the head-mountable computing device. The received data may include the identification information of the medical devices that the wearer is operated, such as the types or the versions of the medical devices, so as to precisely identify the activity of the wearer, i.e. on which medical devices the wearer is operated.

It is understood that the activity identification may be achieved by combining the analysis of the video/image data, the audio/acoustic data and the data received from medical devices on which the wearer is operated.

The activities in the LUT may be pre-categorized into two different types of activity, i.e. the activity that may need assistance information and the activity that may not need any assistance information. For the activity that may need assistance information, the predefined database may include the needed assistance information for each activity. The LUT may define the types of assistance information for each activity. The types of assistance information may include, but not limited to, one or more physiological parameters of an object, the medical images with different modalities of an object, etc. If the wearer's activities cannot be identified, then the method may be triggered to revert back to step 204.

Upon identifying the activity of the wearer of the head-mountable computing device 100, the processor 110 subsequently in step 208 checks whether or not the identified activities may need user assistance information. If it is confirmed that the identified activity may need assistance information, then the method may be triggered to step 210, namely providing assistance information to the wearer by displaying such information on the display module 106. If it is confirmed that the identified activity may not need any user assistance information, then the method may be triggered to step 212, namely hiding or removing all information displayed on the display module 106.

After displaying the assistance information on the display module 106 in step 210 or removing information displayed on the display module 106 in step 212, the method may revert back to step 204 to capture another video data or audio data ore receive another data derived from the one or more medical devices to re-identify the activity of the wearer, or may terminate in step 214.

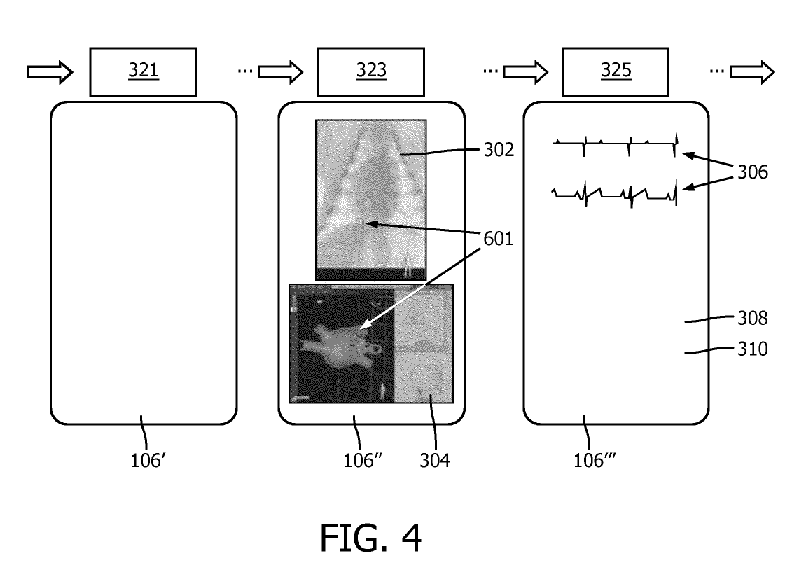

FIG. 4 schematically depicts an aspect of the head mountable computing device of FIG. 2, further showing an example of the wearer's assistance information displayed on the display module 106 for a wearer's activity of performing a catheter ablation operation with interventional X-ray.

The catheter ablation is a procedure to remove or terminate a faulty pathway from sections of the hearts, and has been increasingly used to treat cardiac arrhythmias such as Atrial fibrillation (AF).

After the preparation of the operation (including the preparation with the patient), the procedure of the catheter ablation 321 may be started. The wearer of the head-mountable computing device 100 may first insert a catheter 601 into a patient body. The catheter insertion activity may be detected by the camera 120 of the head-mountable device 100. The detected image may include objects such as the “catheter” and the “hands” of the wearer which may indicate the action of inserting. The detected image may be analysed by the image processing unit 132 of the processor 110. The processor 110 may further compare the identified objects “catheter” and “hands” against a list of objects in a LUT corresponding to a list of activities. As the objects “catheter” and “hands” in the LUT correspond to a catheter insertion activity, then a match is found and the catheter insertion activity is identified. The catheter insertion activity may also be identified based on the objects “catheter” and/or “hands” detection in combination with hand movement pattern analysis. As it is already defined in the LUT that no assistance information is needed for the catheter insertion activity, therefore all the assistance information in the display model 106′ is automatically hidden or removed from the display module such that no assistance information is displayed on the display module 106′, as is shown in the left portion of FIG. 4. This may enable the wearer to focus on inserting the catheter into the patient's body. Alternatively, the wearer may also choose to show some relevant assistance information as they preferred, for example, by pressing the button 122 or sending audio commands to the head-mountable computing device 100.

Before starting ablation, the wearer may need to start the catheter navigation procedure 323, that is, to navigate the catheter tip to the right location in the heart of the patient. The navigation and mapping of the catheter may be normally done by using an X-ray image 302 and a pre-build heart model 304. The wearer may start and stop generating the X-ray image 302 by pressing a pedal with his/her foot. This action may not be detectable by the camera sensors. By connecting or communicating the X-ray system with the head-mountable computing device 100, the data including the identification information of the X-ray machine and the catheter 601 may be transmitted to the head-mountable computing device 100. Based on the received data, the processor 110 may identify that the wearer is currently operating on an X-ray machine as well as a catheter. Based on a predefined rule in the LUT, the X-ray image 302 and the pre-build heart model 304 for indicating the position of the catheter may be shown on the display module 106″, as is shown in the middle portion of FIG. 4. The wearer may observe the current position of the catheter 601 in the patient's body through the X-ray image 302 and the pre-build heart model 304.

After the catheter tip 601 is inserted in the right position of the patient's body for RF ablation, the wearer may start the RF ablation procedure 325, i.e., applying energy to the heart tissue. The start and stop of ablation may be difficult to detect solely with the sensors. By connecting or communicating the catheter 601 to the head-mountable computing device 100, the activity of starting and stopping the RF ablation may be detected. During the process of ablation, the wearer may need to monitor whether the ablation is enough or not, that is, causing good “transmural” lesion in the heart wall. In the current clinical practice, the physician monitors the ECG signals of the patient. From the change in the ECG (Electrocardiogram, or Electrocardiography) signals, the physician can decide whether the ablation is enough or not.

When the processor 110 detects that the wearer starts an RF ablation, based on a predefined rule in the LUT, an ECG or electrograms signals 306, or other assistance information that may indicate the progress of ablation, may be displayed on the display module 106′″, as is shown in the right portion of FIG. 4.

The pressure between catheter tip and heart wall is also often used for monitoring the progress of RF ablation. Based on a predefined rule in the LUT, a pressure indicator 308 may also be displayed on the display module 106′″ during the ablation procedure, as is shown in the right portion of FIG. 4.

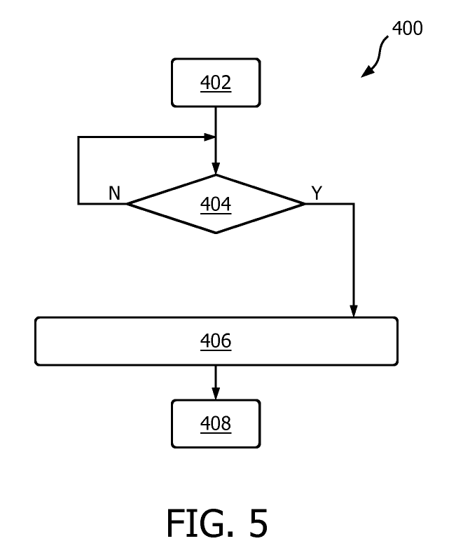

Alternatively, a duration of ablation indicator 310 may also be displayed on the display module 106′″, as is shown in the right portion of FIG. 4. More critically, if the duration of ablation is longer than a pre-defined threshold T, the duration of ablation indicator 310 may be highlighted, e.g. using different color/brightness or different font/format, in order to give the physician an alert. The parameter T may be dependent on the location of catheter tip 601 in the heart, normally the parameter T equals 10 seconds. FIG. 5 shows a flow chart of a second embodiment of a method according to the present invention.

The method 400 starts in step 402 with receiving a starting command regularly triggered by the processor 110. The interval between the two starting commands triggered by the processor 110 may be predetermined by the wearer of the head-mountable computing device 100. The method 400 then proceeds to step 404, namely the processor 110 decides whether a vital sign signal or data of a patient is higher than a predefined threshold. If so, then the method 400 is triggered to step 406, namely displaying a warning message on the display module 106. If not, then the method 400 is reverted back to step 404. After displaying the warning message on the display module 106 in step 406, the method may revert back to step 404 or may terminate in step 408.

Aspects of the present invention may be embodied as a wearable computing device, method or computer program product. Aspects of the present invention may take the form of a computer program product embodied in one or more computer-readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. Such a system, apparatus or device may be accessible over any suitable network connection; for instance, the system, apparatus or device may be accessible over a network for retrieval of the computer readable program code over the network. Such a network may for instance be the Internet, a mobile communications network or the like. More specific examples (a non-exhaustive list) of the computer readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of the present application, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out the methods of the present invention by execution on the processor 110 may be written in any combination of one or more programming languages, including an object oriented programming language such as Java, Smalltalk, C++ or the like and conventional procedural programming languages, such as the “C” programming language or similar programming languages. The program code may execute entirely on the processor 110 as a stand-alone software package, e.g. an app, or may be executed partly on the processor 110 and partly on a remote server. In the latter scenario, the remote server may be connected to the head-mountable computing device 100 through any type of network, including a local area network (LAN) or a wide area network (WAN), or the connection may be made to an external computer, e.g. through the Internet using an Internet Service Provider.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions to be executed in whole or in part on the processor 110 of the head-mountable computing device 100, such that the instructions create means for implementing the functions/acts specified in the flowchart and/or block diagram block or blocks. These computer program instructions may also be stored in a computer-readable medium that can direct the head-mountable computing device 100 to function in a particular manner.

The computer program instructions may be loaded onto the processor 110 to cause a series of operational steps to be performed on the processor 110, to produce a computer-implemented process such that the instructions which execute on the processor 110 provide processes for implementing the functions/acts specified in the flowchart and/or block diagram block or blocks. The computer program product may form part of a head-mountable computing device 100, e.g. may be installed on the head-mountable computing device 100.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The invention can be implemented by means of hardware comprising several distinct elements. In the device claim enumerating several means, several of these means can be embodied by one and the same item of hardware. The mere fact that certain measures are recited in mutually different dependent claims does not indicate that a combination of these measures cannot be used to advantage.

1. A head-mountable computing device comprising:
at least one display module;
an interface adapted to:
receive signals and/or data acquired by one or more sensors; and
a processor adapted to:
identify the wearer's activity based on the received signals and/or data acquired by the one or more sensors; and
control said at least one display module to display assistance information on said display module corresponding to the identified wearer's activity,
wherein the processor is further adapted to control said at least one display module to hide information displayed on said display module corresponding to the identified wearer's activity.

2. The head-mountable computing device according to claim 1, wherein the interface is adapted to receive data generated by one or more medical devices indicating the type of medical devices operated by a wearer of the head-mountable computing device;
wherein the processor is adapted to identify the wearer's activity based on the received signals and/or data acquired by the one or more sensors in combination with data received from the one or more medical devices indicating the type of medical devices operated by the wearer of the head-mountable computing device.

3. The head-mountable computing device according to claim 1, wherein the processor is adapted to control said at least one display module to display a warning message on said display module based on a predefined threshold of the one or more received data.

4. The head-mountable computing device according to claim 1, wherein the data received from the one or more medical devices comprises the identification information of the medical devices.

5. The head-mountable computing device according to any claim 1, wherein the medical device is a catheter or an interventional X-ray.

6. The head-mountable computing device according to claim 5, wherein the one or more sensors are part of the head-mountable computing device, and the one or more sensors are one of imaging sensor, microphone or accelerometer.

7. The head-mountable computing device according to claim 1, wherein the assistance information displayed on said display module includes the duration of an operation activity that the wearer is operated on said one or more medical devices.

8. The head-mountable computing device according to preceding claim 7, wherein the warning message is displayed on the display module when the duration of the operation activity is longer than a predefined value.

9. A method for displaying information on the head-mountable computing device of claim 1, the method comprising:
receiving signals and/or data acquired by one or more sensors;
identifying the wearer's activity based on the received signals and/or data acquired by the one or more sensors;
displaying assistance information on said display module corresponding to the identified wearer's activity.

10. The method of claim 9, further comprising:
receiving one or more signals generated by one or more medical devices indicating the type of medical devices operated by a wearer of the head-mountable computing device
identifying the wearer's activity based on the received signals and/or the data acquired by the one or more sensors in combination with the data received from the one or more medical devices indicating the type of medical devices operated by the wearer of the head-mountable computing device.

11. The method of claim 9, further comprising:
displaying a warning message on said display module based on a predefined threshold of the one or more received signals.

12. The method of claim 9, wherein the assistance information displayed on said display module includes the duration of an operation activity that the wearer is operated on said one or more medical devices, and a warning message is displayed on the display module when the duration of the operation is longer than a predefined value.

13. A computer program product comprising program code means for causing a computer to carry out the steps of the method as claimed in claim 1 when said computer program is carried out on the computer.

14. The head-mountable computing device according to claim 1, wherein the processor is further adapted to control said at least one display module to hide information displayed on said display module corresponding to the identified wearer's activity.

15. The method of claim 10, further comprising:
hiding information displayed on said display module corresponding to the identified wearer's activity.
